(19) United States
(12) Patent Application Publication
(10) Pub. No.: US 2003/0225049 A1
(43) Pub. Date: Dec. 4, 2003

(54) Method of treatment for increasing the number of NMDA receptors in a mammalian brain

(75) Inventor: Monique Vincens, Paris (FR)

Correspondence Address: LARSON & TAYLOR, PLC, 1199 NORTH FAIRFAX STREET, SUITE 900, ALEXANDRIA, VA, 22314, US

(73) Assignee: UNIVERSITE PARIS VI

(21) Appl. No.: 10/441,276

(22) Filed: May 20, 2003

Related U.S. Application Data
(63) Continuation-in-part of application No. 09/856,261, filed on Sep. 24, 2001, now abandoned.
Application No. 09/856,261 is a 371 of international application No. PCT/FR99/02848, filed on Nov. 19, 1999.

(30) Foreign Application Priority Data
Nov. 20, 1998 (FR) 98 14669

(51) Int. Cl. A61K 31/57

(52) U.S. Cl. 514/178

(57) ABSTRACT

The invention concerns the use of DHEAS for making a pharmaceutical composition designed to increase the number of NMDA receptors in a mammal's brain. A method is also provided for increasing the number of NMDA receptors in the brain of a mammal by administering to the mammal a pharmaceutical composition containing dehydroepiandrosterone sulfate, wherein the mammal is a human being of from 40 to 60 years of age and wherein the dehydroepiandrosterone sulfate is administered for a period of at least 20 days.

### CROSS-REFERENCE TO RELATED APPLICATIONS

The present application is a continuation-in-part application of U.S. Ser. No. 09/856,261, filed May 18, 2001, now abandoned.

### FIELD OF THE INVENTION

The present invention relates to a method of treatment comprising the administration of dehydtoepiandrosterone sulfate (DHEAS) in the form of a pharmaceutical composition to a mammalian subject in order to increase the number of NMDA (N-methyl-D-aspartate) receptors in the brain of said subject.

### BACKGROUND OF THE INVENTION

Dehydroepiandrosterone and its sulfate, i.e. DHEAS, are steroid hormones which are mainly synthesized in the adrenal cortex. However, the demonstration of de novo synthesis in the brain suggests that these steroids are active in the central nervous system.

In parallel with the demonstration of this synthesis of steroids in the brain, it was also shown that some steroids of the pregnane or androstrane series were able to modulate the GABA-A (gamma aminobutyric acid) membrane receptor and to possess, in the same manner as all the other modulators of the GABA-A receptor complex which are known so far, a binding site on this receptor (Majewska et al., Science (1986) 232, 1004-1007; Vincens et al. Eur. J. Pharmacol. (1989) 168, 15-21; Sieghart TIPS (1992), 13, 446-450).

Some of the steroids, such as pregnanolone or allopregnanolone, have been characterized as being agonists of the GABA-_{A} receptor (Vincens et al. Naumyn—Schmiedeberg's Arch. Pharmacol. (1992) 346: 523-526), while others, such as pregnenolone sulfate or DHEAS, behave as antagonists (Majewska et al. Neurosci. Lett. (1988) 90, 279-284; Mienville et al. Brain Res. (1989) 489, 190-194; Majewska et al. Brain Res. (1990) 526, 143-146).

However, these two neurosteroids, i.e. pregnenolone sulfate and DHEAS (Wu et al. Mol. Pharmacol. (1991) 40, 333-336; Park-Chung et al. Mol. Pharmacol. (1997) 52, 1113-1123; Debonnel et al. J. of Endocrinol. (1996) 150, S33-S42), could be modulators of the NMDA receptor. The NMDA receptor is implicated in the learning and memorizing faculties (Mondadori et al. Exp. Brain Res. (1989) 75: 449-456).

Various observations suggest that DHEAS may act on memory and also on cerebral aging. Thus, Roberts et al. in Kalimi M and Regelson W (eds) 1990 p13-42 (Berlin: Walter de Gruyter “The Biological Role of DHEA”) demonstrated that administering DHEA or DHEAS orally or subcutaneously to mice had the effect of increasing long-term memory and decreasing amnesia. However, the mechanism by which DHEAS acts in order to improve memory remains unexplained.

Similarly, U.S. Pat. No. 5,556,847, in the name of Johnson et al., indicates that DHEAS and pregnenolone sulfate have an inhibitory action on GABA-_{A} receptors and that they facilitate the action of NMDA receptors in mice.

Furthermore, a physiological decrease in the plasma levels of DHEA and DHEAS in both men and women has been observed during the course of aging (Bélanger et al. J. Clin. Endo. Metab. (1994) 79 1086-1090—Lamberts et al. Science (1997) 278, 419-423).

The number of NMDA receptors has been observed to decrease during the course of cerebral aging in the rat (Tamaru et al. Brain Res. (1991) 542, 83-90), in the mouse (Cohen et al. Brain Res. (1992) 584, 174-180), in elderly human subjects, as well as in the brains of certain patients suffering from Alzheimer's disease (Ulas et al. Neuroscience (1992) 49, 45-61; Greenamyre et al. J. Neurochem. (1987) 48, 543-551; Jansen et al. Neuroscience (1990) 39, 613-627; Mouradian et al. Neurosci. Lett. (1988) 93, 225-230; Penney et al. J. Neurol. Neurosurg., and Psychiatry (1990) 53, 314-320).

It would be very advantageous to prevent the physiological decrease in NMDA receptors which occurs during cerebral aging.

### SUMMARY OF THE INVENTION

The applicant has studied the effect of DHEAS on glutamate receptors, in particular NMDA receptors, in adult male rats and has surprisingly and unexpectedly observed an increase in the number of these receptors in various regions of the brain when the rats are treated for a prolonged period of time with DHEAS.

The applicant has succeeded in increasing the number of NMDA receptors, hence offsetting the physiological decline in the number of these receptors which occurs with age, by administering DHEAS to mammalian subjects.

### BRIEF DESCRIPTION OF THE DRAWING FIGURES

FIGS. 1 to 3 demonstrate the effect of DHEAS on the NMDA receptors in different parts of the brain.

Thus, FIGS. 1 and 2 show the autoradiographic distribution of the [³H]-MK801-labeled NMDA receptors in the rat brain. FIG. 3 shows that the highest density of the labeled sites is always found in the hippocampus, at the level of the CA1 layers and the dentate gyrus.

The different histograms in FIG. 3 show optical density as a function of the brain regions.

FIGS. 3A to 3G show the labeling of the NMDA receptors [³H]-MK801 at different levels.

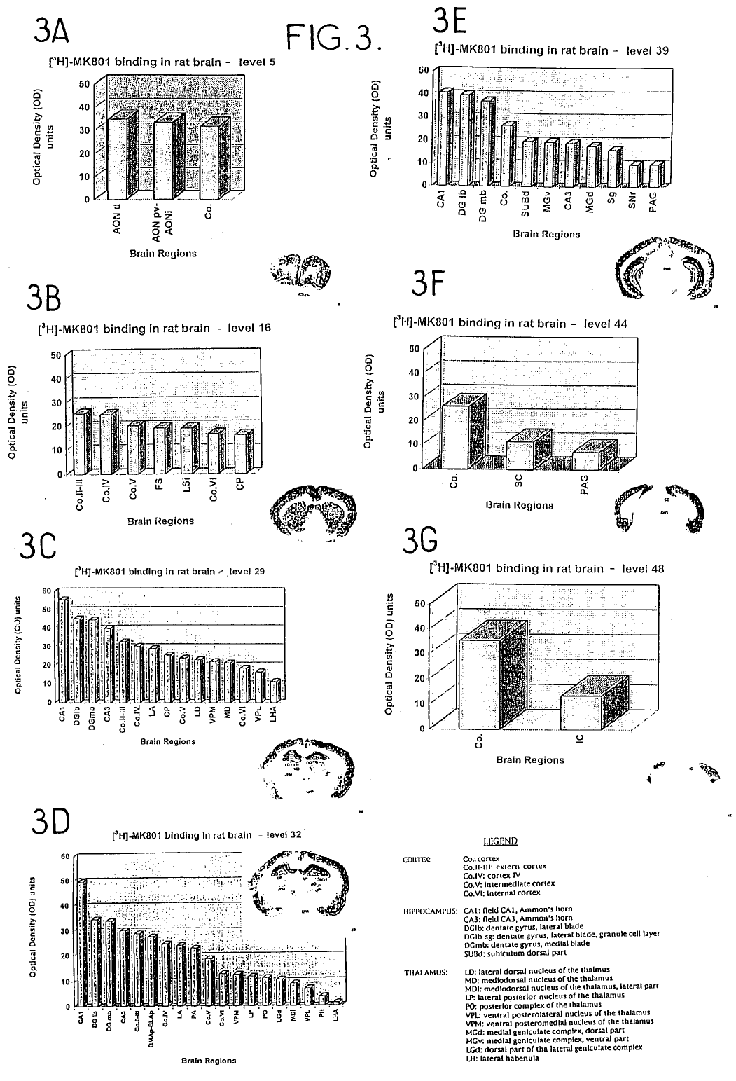

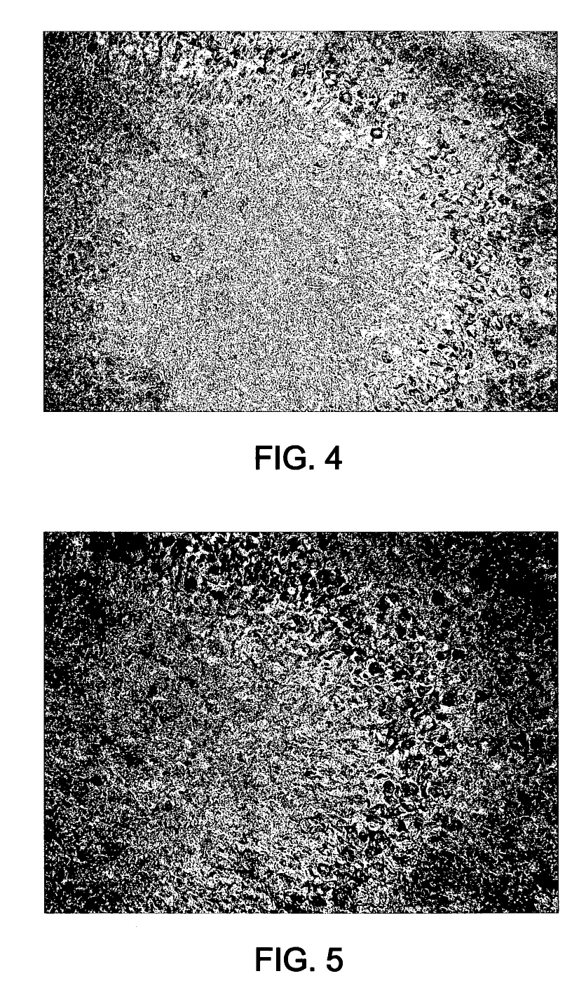

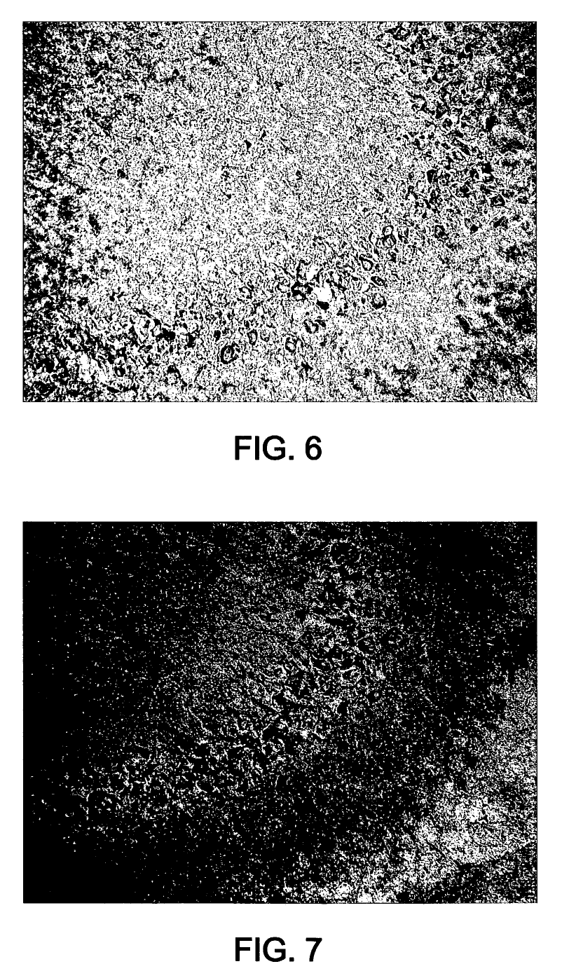

Thus, FIG. 3A shows the distribution and the level of the NMDA receptors at level 5, with FIG. 3B showing these parameters at level 16, FIG. 3C at level 29, FIG. 3D at level 32, FIG. 3E at level 39, FIG. 3F at level 44 and FIG. 3G at level 48.

The following abbreviations are used in these figures:

HYPOTHALAMUS:

LHA: lateral hypothlamic area

AMYGDALA:

LA: lateral nucleus of the amygdala

BLAp: basolateral nucleus of the amygdala posterior part

BMAp: basomedial nucleus of the amygdala

PA: posterior nucleus of the amygdala

SEPTAL REGION:

LSI: lateral septal nucleus, immediate part

CORPUS STRIATUM:

CP: caudoputamen

FS: fundus of the striatum

CORTEX:

Co.: Cortex

Co. II-III:external cortex

Co. IV: cortex IV

Co. V: intermediate cortex

Co. VI: internal cortex

HIPPOCAMPUS:

CA1: Meld CA1, Ammon's horn

CA3: Meld CA3, Ammon's horn

DG1b: dentate gyrus, lateral blade

DG1b-sg: dentate gyrus, lateral blade granule cell layer

DGmb: dentate gyrus, medial blade

SUbd: subiculum dorsal part

THALAMUS:

LD: lateral dorsal nucleus of the thalamus

MD: mediodorsal nucleus of the thalamus

MD1: mediodorsal nucleus of the thalamus lateral part

LP: lateral posterior nucleus of the thalamus

PO: posterior complex of the thalamus

VPL: ventral posterolateral nucleus of the thalamus

VPM: ventral posteromedial nucleus of the thalamus

MGd: medial geniculate complex, dorsal part

MGv: medial geniculate complex, ventral part

Lgd: dorsal part of the lateral geniculate complex

LH: lateral habenula

FIGS. 4 to 7 show immunohistochemical studies of antibodies binding to control and DHEAS-treated portions of rat hippocapmus for subunit NR1 of the NMDA receptor. Thus, FIGS. 4 and 5 show control and DHEAS-treated sections of rat hippocampus treated for 30 days, whereas FIGS. 6 and 7 show control and DHEAS-treated sections of rat hippocampus treated for 20 days.

### DETAILED DESCRIPTION OF THE PREFERRED EMBODIMENTS

The present invention thus relates to a method for increasing the number of NMDA receptors in the brain of a mammal comprising the administration of a pharmaceutical composition containing DHEAS to said mammal.

The method according to the present invention is advantageously targeted at a population of human subjects between the ages of about 40 and 60 years.

In accordance with the invention, the pharmaceutical composition is administered in a dose, and for a period of time, which are sufficient for this administration to lead to an efficacious increase in the number of NMDA receptors.

In a general manner, this dose is between 0.001 and 0.10 g/kg of the body weight of the patient, preferably between 0.002 and 0.05 g/kg, and even more preferably between 0.003 and 0.03 g/kg of body weight.

According to a preferred embodiment of the invention, the pharmaceutical composition comprises between 2 mg and 5 g of DHEAS, preferably 20 mg and 2 g, and even more preferably 50 mg and 1.5 g.

The duration of treatment with DHEAS is preferably of at least 5 days, more preferably at least 20 days and even more preferably at least 30 days.

The pharmaceutical composition may be in the form of tablets, pills, gelatin-coated pills, suppositories, capsules, ampoules, etc. The preferred pharmaceutical form is the tablet form.

The invention also relates to the use of DHEAS for preventing the physiological decrease in NMDA receptors with age.

The invention will be more clearly understood with the aid of the non-limiting examples which follow.

### EXAMPLE 1

1.1 Labeling the NMDA Receptors with the Ligand [³H]-MK801

The NMDA receptors in the rat brain were labeled by means of the quantitative autoradiography technique, thereby making it possible to visualize and quantify the number of receptors in different regions of the brain.

The radioactive ligand which makes it possible to specifically recognize the sites of the NMDA receptors is tritium-labeled dizocilpine (MK801), i.e. [³H]-MK801 (Kemp et al. TINS (1987) 294-298).

Male rats (Wistar) weighing between 200 and 220 g were decapitated. Their brains were rapidly removed and placed in liquid isopentane at a temperature of −40° C. The brains were then stored at −80° C. until used.

Brain sections having a thickness of 20 pm were obtained using a Frigocut 2800® cryostat, which is marketed by Reichert Jung.

The brain sections were preincubated in a 50 mM Tris-acetate, pH 7.4, buffer at 4° C. for 30 minutes. They were then dried rapidly at room temperature.

The sections were then incubated with the radioligand [³H]-MK801, sold by New England Nuclear (Boston, Mass., USA) and having a specific activity of 24 Ci/mmol, at a concentration of 5 nM in the abovementioned buffer at 4° C. for a period of 4 hours.

Control sections were incubated with MK801, which was present at a concentration of 10⁻⁵ M and which was not labeled with tritium, in order to determine non-specific binding.

All the sections were then rinsed for 5 seconds and then for 90 minutes in the same cold buffer.

The sections were dried at room temperature and then exposed on tritium-hyperfilm films (Amersham) at 4° C. in the dark for 4 weeks in autoradiography cassettes.

The films were developed for 2 minutes in a Kodak D19 developer and then fixed in Kodak Rapid fixer and finally rinsed for an hour in running water.

The autoradiographic labeling was quantified by densitometry using the RAG-200 software sold by BIOCOM (Les Ulis, France), and the binding was measured by relative optical density.

1.2 Mapping the [³H]-MK801-Labeled NMDA Receptors in the Rat Brain

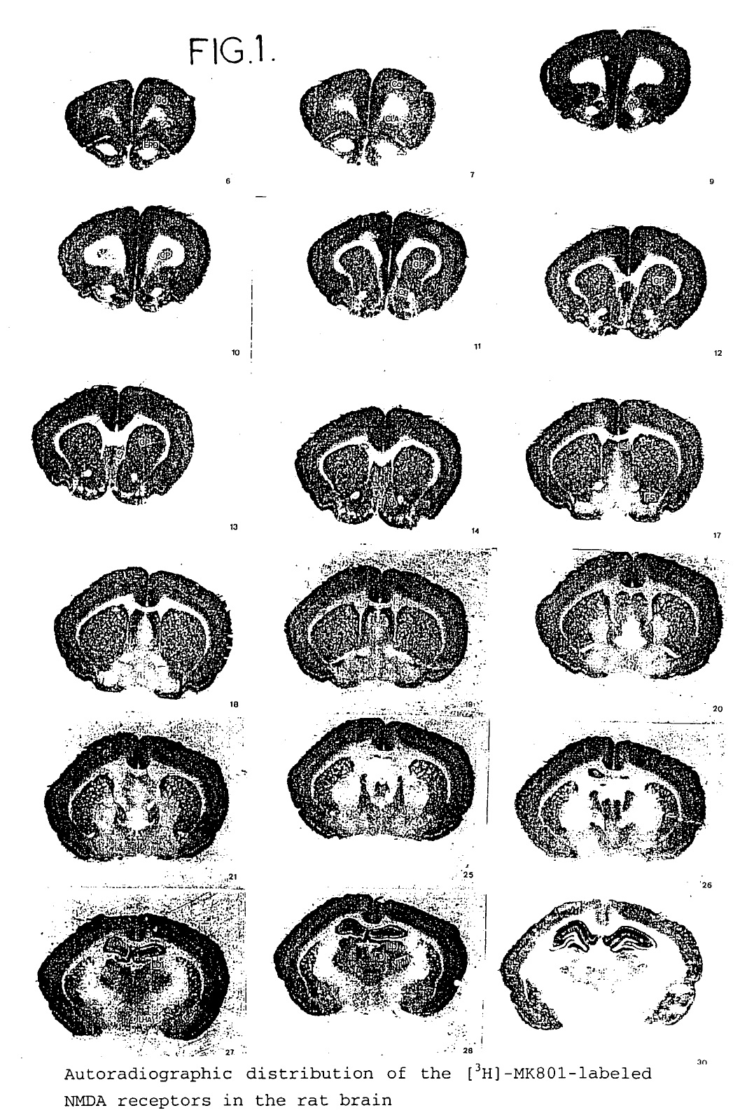

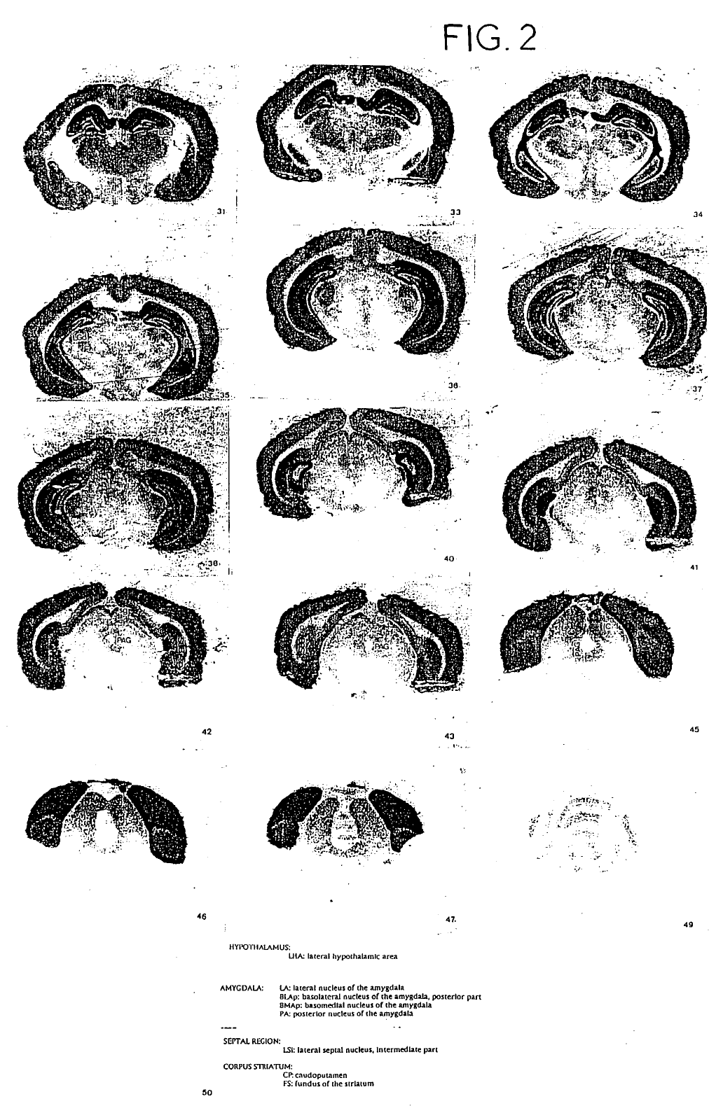

The [³H]-MK801-labeled NMDA receptors in the rat brain were mapped using the above-described autoradiography protocol (see FIGS. 1 and 2).

These results show that, whatever the level studied, the highest density of labeled sites is always found in the hippocampus, at the level of the CA1 layers and the dentate gyrus. These layers are particularly implicated in memory function (see FIG. 3).

### EXAMPLE 2

Effect of Treatment with DHEAS on the NMDA Receptors in the Rat Brain

30 mg of DHEAS were administered/kg, intraperitoneally and twice daily, to non-castrated male Wistar rats for 5 days. The rats were sacrificed on the 5th day, 30 minutes after the last injection of DHEAS.

Table I below shows that this treatment increases the number of apparent [³H]-MK801-labeled NMDA receptor sites, as measured by optical density (OD), in certain regions of the brain.

TABLE I
Effect of treatment with DHEAS on the number of
[³H]-MK801 sites in the rat brain.

| | | Rats treated |
|---|---|---|
| | Control rats | with DHEAS |
| Regions | OD | OD |
| CA₁ | 44.67 ± 3.63 | 54.01 ± 4.05* |
| CA₃ | 28.84 ± 3.15 | 33.37 ± 4.94* |
| DG1b | 37.45 ± 3.95 | 42.15 ± 4.31* |
| DGmb | 36.17 ± 4.15 | 43.13 ± 3.53* |
| Cortex | 21.32 ± 1.70 | 24.88 ± 2.73 |

*< 0.001

The apparent [³H]-MK801-binding sites are measured by optical density OD for the total specific binding of [³H]-MK801 in the cortex and different regions of the hippocampus: CA₁; CA₃; lateral blade of the dentate gyrus, DG1b, and the median blade of the dentate gyrus, DGmb.

These results show that a chronic 5-day treatment in the male rat increases the apparent number of [³H]-MK801-binding sites in the different regions which were quantified.

### EXAMPLE 3

Increase in NR1 and NR2 Subunits of NMDA Receptors in Rat Brain as a Result of Treatment with DHEAS

Male Wistar rats weighing 200-220 g (IFFA CREDO, Paris) were housed at 21° C. with a standard 12 h light/12 h dark cycle and with food and water being freely available. The animals received intraperitoneal (i.p.) injections of either the vehicle dimethyl sulfoxide (DMSO; 0.2 ml/250 g body weight(b.w.) or dehydroepiandrosterone sulfate (DHEAS; 30 mg/kg b.w. dissolved in DMSO (0.2 ml/250 g b.w)) twice daily, at 8 a.m. and 6 p.m., for a period of either 5, 20 or 30 days. The last injection was given at 8 a.m. of the 5th, 20th or 30th day. All the animals were killed 2 hours after the last injection. Six groups of animals (ten animals per group) were used. Three control groups received DMSO alone for a period of 5, 20 or 30 days respectively, and, in a similar manner, three groups received DHEAS treatment for a period of 5, 20 or 30 days respectively.

The rats were killed by decapitation and the brains were rapidly removed from the skull, quickly frozen in liquid isopentane at −40° C., and kept at −80° C. until sectioned. The frozen brains were cut into 20 um thick sections in a cryostat (Frigocut 2800, Reichert Jung). Coronal sections were serially cut through the midbrain (levels 18-24, Paxinos and Watson's atlas), mounted on 2% gelatin-coated microscope slides, and kept at −20° C. until use.

The Kit Elite Vectastain (Abcys-France) was used for the immunohistochemical studies. The antibodies against NMDA R1 and NMDA R2 A/B receptors were purchased from Chemicon International, Inc (USA).

The mounted sections were brought to room temperature for 1 h and then fixed in Acetone for 5 nm. The sections were rinsed, first in tap water (5 nm) and then twice 5 nm in Phosphate Buffered Saline (PBS, Sigma) 0.1 M, ph 7.4, and were then preincubated for 20 nm in the same buffer containing 0.15% horse serum. Sections were incubated for 24 h at 4° C. in rabbit polyclonal anti-NMDAR1 or anti-NMDAR2 A/B receptors, diluted 1:1000 in PBS. After rinsing in PBS twice for 5 nm, the sections were subsequently incubated for 30 nm in biotinylated horse anti-body immunoglobulins diluted in PBS at 1:50, then rinsed in PBS (twice 5 nm) and incubated for 30 nm in peroxidase-conjugated streptavidin, diluted 1:50. The sections were washed in PBS (twice 5 nm) and the peroxidase reaction product was visualized by the glucose oxidase-nickel-DAB method (Shu. S., Ju, G and Fan L, 1988, Neurosci. Lett. 85,163-171). All sections were mounted on gelatin-coated slides, dehydrated and coverslipped with Eukitt (Labonord-France). Immunostaining controls included deletion of the primary antibodies during the procedure and showed no immunoreaction in accordance with previous specificity studies.

The results showed a clear increase in the number of NR1 subunits after 5, 20 and 30 days of treatment with DHEAS. The increase can be quantified by the SCION program developed at the National Institutes of Health (USA). Morphometrie was performed using a microscope M2FL₃ Lexca connected to a camera and an image analyser (Shu et al 1988).

After DHEAS treatment (5, 20 and 30 days), an increase of 15 to 20% of NR₁ subunits was observed in the hippocampus area. A slight increase was also observed for NR₂ subunits.

FIGS. 4 and 5 respectively show control and DHEAS-treated segments of rat hippocampus treated at 30 days; whereas FIGS. 6 and 7 respectively show control and DHEAS-treated segments of rat hippocampus treated at 20 days. Both sets of figures clearly demonstrate the increase in the NR1 subunit at 20 days and at 30 days of treatment with DHEAS.

What is claimed is:

1. A method for increasing the number of NMDA receptors in the brain of a mammal comprising administering to said mammal a pharmaceutical composition containing dehydroepiandrosterone sulfate, wherein the mammal is a human being of from 40 to 60 years of age and wherein the dehydroepiandrosterone sulfate is administered for a period of at least 20 days.

2. The method according to claim 1 wherein the dehydroepiandrosterone sulfate is administered for a period of at least 30 days.

3. The method according to claim 1 wherein the dose of dehydroepiandrosterone sulfate in the pharmaceutical composition is between 0.001 and 0.10 g/kg of the body weight of the mammal.

4. The method according to claim 1 wherein the dose of dehydroepiandrosterone sulfate in the pharmaceutical composition is between 0.002 and 0.05 g/kg of the body weight of the mammal.

5. The method according to claim 1 wherein the dose of dehydroepiandrosterone sulfate in the pharmaceutical composition is between 0.003 and 0.03 g/kg of the body weight of the mammal.

6. The method according to claim 1 wherein the pharmaceutical composition comprises between 2 mg and 5 g of dehydroepiandrosterone sulfate.

7. The method according to claim 1 wherein the pharmaceutical composition comprises between 20 mg and 2 g of dehydroepiandrosterone sulfate.

8. The method according to claim 1 wherein the pharmaceutical composition comprises between 50 mg and 1.5 g of dehydroepiandrosterone sulfate.

9. The method according to claim 1 wherein the pharmaceutical composition is in a form selected from the group consisting of tablets, pills, gelatin-coated pills, suppositories, capsules or ampoules.

10. The method according to claim 1 wherein the pharmaceutical composition is in a form of tablets.

11. A method for prophylaxis of the physiological decrease in the number of NMDA receptors which occurs with age in the brain of a mammal, comprising administering to said mammal a pharmaceutical composition containing dehydroepiandrosterone sulfate, wherein the mammal is a human being of from 40 to 60 years of age and wherein the dehydroepiandrosterone sulfate is administered for a period of at least 20 days.

12. The method according to claim 11, wherein the dehydroepiandrosterone sulfate is administered for a period of at least 30 days.
